(19) United States
(12) Patent Application Publication
(10) Pub. No.: US 2013/0345140 A1
(43) Pub. Date: Dec. 26, 2013

(54) Basement Membrane Compositions and Applications Thereof

(71) Applicant: Roy Ogle, Norfolk, VA (US)

(72) Inventor: Roy Ogle, Norfolk, VA (US)

(73) Assignee: LifeNet Health, Virginia Beach, VA (US)

(21) Appl. No.: 13/818,904

(22) PCT Filed: Aug. 25, 2011
(86) PCT No.: PCT/US11/49192

§ 371 Date: Sep. 3, 2013

Related U.S. Application Data
(60) Provisional application No. 61/376,966, filed on Aug. 25, 2010.

(51) Int. Cl. C12N 5/0775; A61K 31/737; A61K 38/17; A61K 38/39

(52) U.S. Cl. CPC C12N 5/0675; A61K 38/39; A61K 31/737; A61K 38/1719; C12N 5/0667
USPC 514/17.2; 435/395; 435/377; 435/398

(57) ABSTRACT

The invention relates to cell support compositions comprising a basement membrane extract isolated from cardiac or smooth muscle. The invention also relates to methods of using the cell support compositions for supporting cellular functions.

### CROSS-REFERENCE TO RELATED APPLICATIONS

This application claims priority to U.S. Provisional Application Ser. No. 61/376,966, filed 25 Aug. 2010, which is incorporated by reference in its entirety.

### BACKGROUND OF THE INVENTION

1. Field of the Invention

The invention relates to cell support compositions comprising a basement membrane extract isolated from cardiac or smooth muscle. The invention also relates to methods of using the cell support compositions for supporting cellular functions.

2. Background

Basement membranes are thin, continuous sheets that separate an epithelium from adjacent stroma and surround nerves, muscle fibers, smooth muscle cells and adipose cells. As shown in many species, basement membranes appear early during embryogenesis and cover the inner cell mass, suggesting a critical role for basement membrane in early development. All basement membranes contain members of 3 extracellular matrix families: laminins, collagen IV and proteoglycans, along with the glycoprotein, nidogen/entactin. The basement membrane is a nanoscale network of filaments that interacts to form a lattice. This network, in part, results from the assembly of independent networks of laminins and collagen IVs, joined together by the nidogens and other associated proteins.

Basement membrane preparations have been used for cell biology research to provide a physiological environment promoting cell attachment, growth, and differentiation. Thus for culturing embryonic and other pluripotent stem cells in vitro, the basement membrane promotes cell growth and can be used to promote the differentiation of certain cell types.

Among the most broadly employed basement membrane preparations is the basement membrane-rich matrix from Engelbreth Holm-Swarm (EHS) murine sarcoma, known by its trade name MATRIGEL™ (BD Biosciences). MATRIGEL™ has been a useful product to facilitate cell growth, development and differentiation for a broad assortment of cell types including stem cells, epithelial cells, endothelial cells, adipocytes, neurons, all forms of muscle cells and their precursors. MATRIGELTM has been used commonly as an alternative to mouse embryonic fibroblast feeder layer to propagate pluripotent stem cells.

However, MATRIGEL™ has limited utility in many applications that arises from its origin as a mouse tumor or limits in the biological activity of the components that comprise it. Many products and processes for making these products require that the products or processes used to make the products be free of animal products. Clearly mouse-derived products are not suitable, for example, for the culture of human cells where the cells are grown to generate biologics products for human use such as antibodies or other proteins. The mouse-derived products are unsuitable for fabrication of tissue engineering scaffolds or other forms of implantation in humans due to concerns of passage of murine pathogens or elicitation of unwanted immune response. Beyond the concern of species difference is that of the composition itself. For example, MATRIGEL™ contains only laminin type I and alpha 1 and alpha 2 chains of collagen type IV. There are 6 chains of type IV collagen and 15 chains of laminin found in human cells, each assumed to impart specific biological functions to the basement membranes in which they occur.

There are undoubtedly components of the basement membrane of each tissue that are unique to that given tissue, although this area of research still requires much greater elucidation. For example, the heart is known not to contain a specific population of stem cells that give rise to cardiomyocytes. Rather, the cardiac tissue contains specific signaling molecules like the chemokines, SDF-1, which recruits circulating stem cells to the heart upon damage. Likewise, the basement membrane composition of tissues like smooth muscle or cardiac muscle constitute a unique signature for each tissue.

### SUMMARY OF THE INVENTION

The invention relates to cell supports compositions comprising a basement membrane extract isolated from cardiac or smooth muscle tissue. The compositions comprise molecules from extracellular matrices including, but not limited laminin, collagen, perlecan, and nidogen/entactin. In some embodiments, the composition may further comprise additional heparan sulfate proteoglycans.

The invention also relates to methods of using the novel compositions for supporting cellular functions, including but not limited to promotion of cell attachment, proliferation, differentiation, or maintenance of the phenotype of differentiated cells, or maintenance of the totipotency, pluripotency, multipotency of stem cells or progenitor cells through multiple culture passages in vitro

The invention also provides methods of applying or injecting the cell support compositions of the present invention in a subject in need of treatment such treatment.

The invention also relates to articles of manufacture comprising the cell support compositions of the present invention. For example, the invention provides for cell culture surfaces comprising the cell support compositions of the present invention.

### BRIEF DESCRIPTION OF THE DRAWINGS

FIG. 1 depicts the protein content of the cell support compositions of the present invention. Equivalent amounts of protein from 4 preparations of human heart basement membrane extract (HBME) from individual donors and MATRIGEL™ (MG) were resolved by electrophoresis on 5% SDS-polyacrylamide gels. The pattern of polypeptides in HBME was very reproducible and distinct from that of MATRIGEL™. The majority of the protein in MATRIGELTM is present as laminin 1—the alpha 1 chain migrates at 400 kD and the beta 1 and gamma 1 chains co-migrate at about 200 kD. A number of the polypeptide bands in the region of 180-400 kD in the HBME samples reacted positively with monospecific antibodies against known laminin chains. Similarly, specific antibodies to type IV collagen (alpha 1 and alpha 2 chains), type I collagen, nidogen/entactin and perlecan were able to bind to the HBME. Nidogen was also present. Tandem mass spec sequencing analysis provided further confirmation of the identity of these polypeptides and identified the presence of several non-basement membrane cytoskeletal proteins, including titin protein, myosin 7 and actinin.

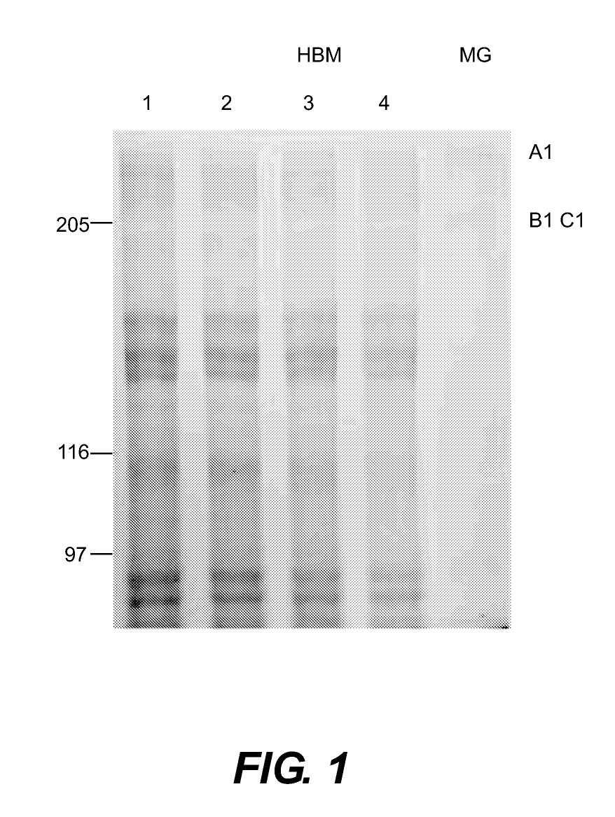

FIG. 2 depicts the attachment of cells to the cell support composition of the present invention. 20,000 of each cell type was allowed to attach to a surface coating of 10 μG/cm² for 1 hour at 37° C. in serum free media. The Table in this figure shows the ratio of attachment to the cell support compositions of the present invention relative to MATRIGEL™. For stem cells and fibroblasts, the cell support compositions of the present invention promote roughly twice the level of attachment as compared to MATRIGEL™.

FIG. 3 depicts the growth and maintenance of plasticity of pluripotent stem cells cultured on the cell support compositions of the present invention and on MATRIGEL™. Either the cell support compositions of the present invention or MATRIGEL™ was coated at 17 μG/cm² were tested for the ability to support the growth and prevent the unwanted differentiation of embryonic and induced pluripotent stem cells in serum-free, chemically defined medium. Mouse embryonic stem cells (D3), mouse induced pluripotent stem cells (W5) or human induced pluripotent stem cells were plated and passaged 2 times on the indicated substrates. Proliferation rates and colony morphology was comparable. When the human induced pluripotent stem cells were stained at second passage using a live cell marker for pluripotency (Tra-1-60, stain alive, Stemgent) the green FITC signal indicates the cells were still in a pluripotent state.

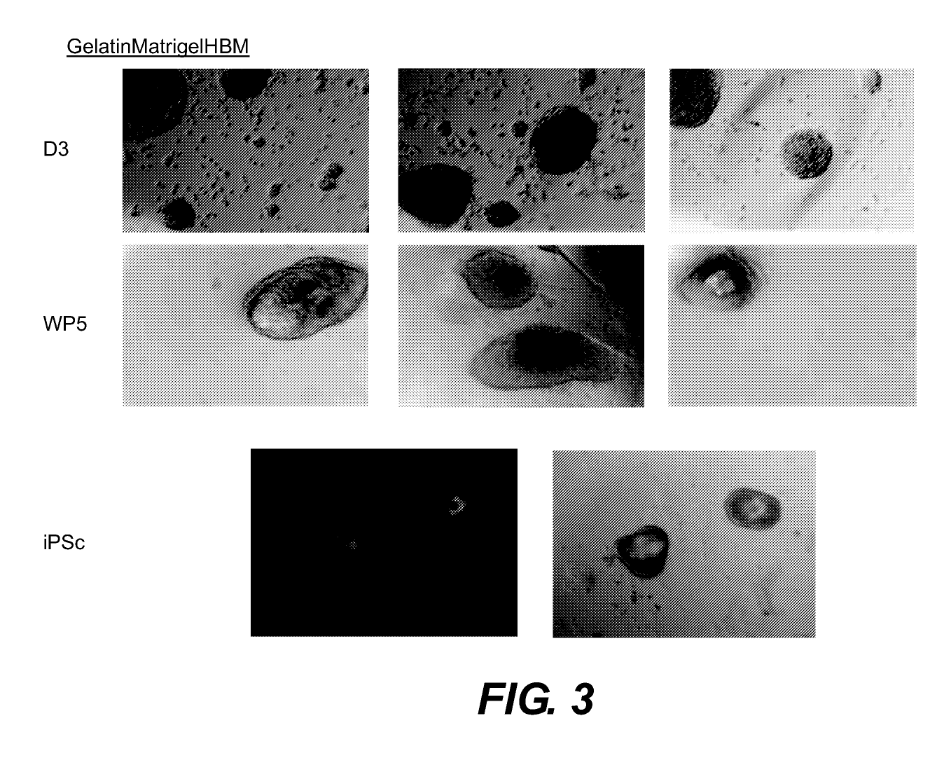

### DETAILED DESCRIPTION OF THE INVENTION

The invention relates to a cell support composition comprising a basement membrane extract isolated from human cardiac or smooth muscle tissues. The compositions comprise molecules from extracellular matrices including, but not limited laminin, collagen, perlecan, and nidogen/entactin. In some embodiments, the compositions may further comprise additional heparan sulfate proteoglycans. The cell support compositions can be obtained from any mammal, such as but not limited to dogs, cats, horses, pigs, cows, non-human and human primates. It is understood that the components of the cell support compositions of the present invention will be species-specific base on the source of the tissue. For example, if the cell surface compositions of the present invention are extracted from human cardiac basement membrane, then the core components of the composition, e.g., laminin, will be human laminin.

If the tissue from which the compositions are extracted is human tissue, the human tissue can, but need not, be obtained from certified tissue banks. The starting material, regardless of the animal source, can also be screened for bioburden, endotoxins and/or the presence of known pathogens. If the material is obtained from a tissue bank, it is likely that the tissue bank would perform the screening process to ensure safety and efficacy. Accordingly, the methods of preparing the cell support compositions of the present invention as disclosed below may or may not include steps of screening the material for bioburden, endotoxins and/or known pathogens. The screening steps may be performed on the starting material or the screening steps may be performed after extraction of the cell support compositions.

The compositions of the present invention comprise laminin type 2 and laminin type 10. In some embodiments, total laminin is present in the cell support compositions of the present invention in an amount from about 5% to about 50% by weight. As used herein, laminin type 2 is well understood in the art and is understood to mean a laminin molecule with an alpha 2 chain, a beta 1 chain and a gamma 1 chain. Laminin 10 is also well understood in the art and is understood to mean a laminin molecule with an alpha 5 chain, a beta 1 chain and a gamma 1 chain. In some embodiments, the compositions of the present invention also comprise one or more additional types of laminins, with these additional types of laminins containing an alpha 2, 3, 4 or 5 chain. Examples of laminins containing alpha chains 2, 3, 4 or 5 include but are not limited to, laminin type 2, laminin type 4, laminin type 5, laminin type 6, laminin type 7, laminin type 8, laminin type 9, laminin type 10, laminin type 11, laminin type 12, laminin type 13, laminin type 14, and laminin type 15. The molecular structure of laminin present in the compositions can be elucidated by using monoclonal antibodies specific for one of the chains the laminin type to isolate the laminin, which can then be analyzed using native gels or chromatography followed by mass spectroscopy and/or SDS-PAGE. One of skill in the art would easily be able to identify the laminin types based on well-known procedures in the art.

The compositions of the present invention do not contain any detectable levels of laminin type 1. In one embodiment, the compositions of the present invention lack detectable levels of laminin type 3. In another embodiment, the compositions of the present invention lack detectable levels of both laminin type 1 and laminin type 3. As used herein, laminin type 1 is well understood in the art and is understood to mean a laminin molecule with an alpha 1 chain, a beta 1 chain and a gamma 1 chain. Laminin type 3 is also well known in the art and is understood to mean a laminin molecule with an alpha 1 chain, a beta 2 chain and a gamma 1 chain. In another embodiment, the compositions of the present invention lack any detectable levels of any laminin that normally contains an alpha 1 chain.

In yet another embodiment, the compositions of the present invention do not contain any detectable levels of laminin type 5. In still another embodiment, the compositions of the present invention lack detectable levels of both laminin type 1 and laminin type 5. In still another embodiment, the compositions of the present invention lack detectable levels of both laminin type 3 and laminin type 5. In still another embodiment, the compositions of the present invention lack detectable levels of laminin type 1, laminin type 3 and laminin type 5. As used herein, laminin type 5 is well understood in the art and is understood to mean a laminin molecule with an alpha 3 chain, a beta 3 chain and a gamma 2 chain. In another embodiment, the compositions of the present invention lack any detectable levels of any type of laminin that normally contains a beta 3 chain and/or a gamma 2 chain.

The compositions of the present invention comprise at least one type of collagen. In one embodiment, the composition comprises type IV collagen. In some embodiments, total collagen is present in the cell support compositions of the present invention in an amount from about 5% to about 50% by weight. In another embodiment, the composition comprises type I collagen. In yet another embodiment, the composition comprises type III collagen. In another embodiment, the composition comprises type XIII collagen. In another embodiment, the composition comprises type XVII collagen. In yet another embodiment, collagen type II is absent from the cell support compositions of the present invention. The types of collagen that are present or absent in the composition can be easily assessed using routine methods in the art. The type IV collagen was identified by its unique molecular mass (180 kD) and reaction with multiple monospecific antibodies. Methods of identifying and quantifying type of collagen are well known in the art, as disclosed, for example in Schnaper, H. W. and Kleinman, H. K., Pediatr. Neprol., 7:96-104 (1993), which is incorporated by reference.

The compositions of the present invention comprise perlecan. Perlecan is a well known protein in the art that is also known as basement membrane-specific heparan sulfate proteoglycan core protein. In some embodiments, perlecan is present in the cell support compositions of the present invention in an amount from about 5% to about 60% by weight. Perlecan is essential for normal vascularization, critical for normal heart development and critical for regulating vascular responses to injuries.

The compositions of the present invention comprise entactin, which is also known as nidogen 1 protein. In some embodiments, entactin is present in the cell support compositions of the present invention in an amount from about 5% to about 50% by weight.

In some embodiments, the compositions of the present invention further comprise titin protein, which is also known as connectin protein. In some embodiments, the amount of titin protein, by weight, present in the cell support compositions of the present invention can be up to about 15%.

In some embodiments, the compositions of the present invention further comprise myosin 7 protein. In some embodiments, the amount of myosin protein, by weight, present in the cell support compositions of the present invention can be up to about 15%.

In some embodiments, the compositions of the present invention further comprise actinin protein. In some embodiments, the amount of actinin protein, by weight, present in the cell support compositions of the present invention can be up to about 7.5%. In still additional embodiments, the compositions of the present invention may comprise one or more of titin, myosin 7 and actinin, including all three molecules.

In select embodiments the compositions optionally comprise signaling molecules. In one embodiment, one or more signaling molecules are naturally present in cardiac or smooth muscle, such that extract composition contains one or more signaling molecules normally present in the natural tissue, i.e., “endogenous signaling molecules.” In another embodiment, one or more signaling molecules can be added to the extract such that the one or more signaling molecules present in the composition are not normally present in the natural tissue, i.e., exogenous signaling molecules. In yet another embodiment, additional levels of one or more signaling molecules that are normally present in the natural tissue can be added to the extract such that the levels of a particular signaling molecule may be higher in the composition than would normally be after the extraction process, i.e., “additional endogenous signaling molecules.” The exogenous signaling molecules or the additional endogenous signaling molecules added to the extracts of the invention need not be from the same animal source as the source of the extracted tissue, e.g., mouse VEGF may be added to human-derived cardiac basement membrane extract. In addition, the exogenous signaling molecules and the additional endogenous signaling molecules added to the extract can be recombinant or isolated from an organism, cell or tissue. As used herein, the term “natural tissue” is used to mean heart muscle or smooth muscle that is either present in an organism or that has been physically removed from the organism, but prior to any processing steps, e.g., enzyme digestion. Tissue that is removed and frozen and has not been subjected to any processing steps, e.g., enzyme digestion and the like, is considered to be “natural tissue” for the purposes of the present invention.

As used herein, a “signaling molecule” is a molecule that either initiates an intracellular signal or signaling pathway or is an intermediate molecule in the signaling or pathway process. A “signal” is a stimulus that creates a cellular response thereto. Signaling molecules can include any molecule that either initiates or participates in the signaling pathway. For example, signaling molecules include but are not limited to growth factors, cytokines and chemokines that may or may not be present in the natural tissue.

Examples of signaling molecules include but are not limited to fibroblast growth factors (FGF), e.g., FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23, epidermal growth factor (EGF), nerve growth factor (NGF), platelet derived growth factor (PDGF-AA, AB and BB), insulin-like growth factors, e.g., IGF-1 and 2, transforming growth factor beta, e.g., TGF-beta 1,2 or 3, vascular endothelial cell growth factor, e.g., VEGF-A, VEGF-B,VEGF-C,VEGF-D, placental growth factor, tumor necrosis factor alpha (TNF-alpha), TNF-beta, bone morphogenetic factors e.g., BMP1, BMP2, BMP3, BMP4, BMP5, BMP6, BMP7, BMP8, BMP9, BMP10, BMP11, BMP12, BMP13, BMP14,and BMP15, Leukemia inhibitory factor (LIF-1), endostatin, angiostatin, thrombospondin, osteogenic protein-1, osteonectin, somatomedin-like peptide, osteocalcin, interferons (IFN), such as but not limited to IFN Type I, IFN Type II and IFN Type III, examples of all of which include but are not limited to IFN-alpha, IFN, beta, IFN-gamma and IFN-omega, interleukins (IL), e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33 and IL-35 and stromal-derived factor (SDF-1) to name a few.

The compositions of the present invention may or may not be polymerized in any setting used. The cell support compositions of the present invention are liquid at temperatures of between about 0° C. and about 8° C. at normal atmospheric pressure. The compositions, however, will polymerize within about 2-10 hours when the temperature is raised to at least about 22° C. or within about 20 minutes to about 1 hours at 37° C. As used herein, the term “polymerize” or “gelation” when used in conjunction with the cell support compositions of the present invention means that the compositions form a gel. The term gel is well understood in the art and, in general, means a cross-linked network of molecules dispersed in a liquid that generally exhibits little to no flow. As used herein, the terms “gelation” and “polymerization” are interchangeable when used in conjunction with the cell support compositions of the present invention.

In some embodiments, the cell support compositions further comprise materials that are added to the compositions. The additional materials may be natural or synthetic, or a combination thereof. Examples of natural materials include, but are not limited to, amino acids, peptides, polypeptides, proteins, carbohydrates, lipids, nucleic acids, glycoproteins, glycosaminoglycans, and proteoglycans. Examples of synthetic materials include, but are not limited to, polymers such as poly (lactic acid) (PLA), polyglycolic acid (PGA), copolymers of PLA and PGA, polycaprolactone, poly (ethylene-co-vinyl-acetate), (EVOH), poly (vinyl acetate) (PVA), polyethylene glycol (PEG) and poly (ethylene oxide) (PEO).

The cell support compositions of the present invention can be used in cell culturing techniques. As used herein, cell culture refers to the maintenance of cells in an artificial environment, commonly referred to as an in vitro environment. The term cell culture is a generic term and may be used to encompass the cultivation not only of individual cells, but also of tissues, organs, organ systems or whole organisms. The cells used in the culture methods disclosed herein can be any prokaryotic or eukaryotic cell. The cell type used in the culture methods disclosed herein need not be from the same species from which the cell support compositions derive. In addition, the cells may be from an established cell line, or they may be primary cells or genetically engineered cells.

The cell support compositions of the present invention can be used in in vitro methods for supporting cell growth and proliferation as well as for sustaining or maintaining the plasticity of stem cells in culture. Accordingly, the invention provides methods of maintaining the plasticity of stem cells with the methods comprising culturing stem cells on a cell culture surface that comprises the cell support compositions of the present invention. In one embodiment, the cell support composition is polymerized prior to culturing the stem cells on the cell surface.

As used herein, a stem cell is used as it is in the art and means a cell that has the ability to divide and give rise to one daughter cell that may be at least partially differentiated and to another daughter cell that retains the developmental potential of the mother cell. As used herein, stem cells can be adult stem cells (ASCs), embryonic stem cells (ESCs), committed progenitor cells, and/or induced pluripotent stem cells (iPSCs).

The terms “cell” and “cell line” may be used interchangeably herein. In one embodiment, stem cells that are attached to cell culture surfaces coated with the cell support compositions of the present invention and are able to maintain their state of plasticity after attachment. Specifically, cells attached on surfaces coated the cell support compositions of the present invention can maintain their state of plasticity for up to at least 2, 3, 4, 5, 6, 7, 8, 9, 10, 11, 12, 13, 14, 15, 20, 25, 30, 35, 40, 45, 50, 55, 60, 65, 70, 75, 80, 85, 90, 95 or 100 passages or even more. As used herein, “state of plasticity” is used to mean the development potential that the cells have, such as, but not limited to, pluripotent, totitpotent, multipotent or unipotent cells. Maintaining a state of plasticity indicates that cell daughter cells, after division, are equally as plastic as the parent cells, e.g., the daughter cells are pluripotent like the pluripotent parent cell. In another embodiment, the cells that are attached to surfaces coated with the cell support compositions of the present invention are able to maintain at least a partial state of plasticity after attachment. Specifically, the cells attached on surfaces coated with the cell support compositions of the present invention can maintain at least a partial state of plascticity for up to at least 2, 3, 4, 5, 6, 7, 8, 9, 10, 11, 12, 13, 14, 15, 20, 25, 30, 35, 40, 45, 50, 55, 60, 65, 70, 75, 80, 85, 90, 95 or 100 passages or even more. As used herein, “partial state of plasticity” is used to mean the development potential that the daughter cells have, such as, but not limited to, pluripotent, totitpotent or unipotent cells. Maintaining at least a partial state of plasticity indicates that cell daughter cells, after division, are not as plastic as the parent cells but nonetheless are still in an undifferentiated state, e.g., the daughter cells are multipotent whereas the parent cell was considered to be pluripotent.

The terms pluripotent, totipotent, multipotent and unipotent are used as they are in the art. Namely, a pluripotent cell is a cell that, depending on environment, has the potential to develop into any type of mature cell found in the organism from which it derived, except germ cells. A totipotent cell is a cell that, depending on environment, has the potential to develop into any type of mature cell found in the organism from which it derived, including germ cells. A multipotent cell is a cell that, depending on environment, has the potential to develop into several different types of mature cell found in the organism from which it derived, usually 2 or more. One example of a multipotent cell is a hematopoietic stem cell. A unipotent cell is a cell that, depending on environment, has the potential to develop into one type of mature cell found in the organism from which it derived. Unipotent cells are not completely differentiated cells, but may be partially differentiated or completely undifferentiated.

The cell support compositions of the present invention can be used in methods for supporting cell growth and proliferation as well as for sustaining or maintaining the state of differentiation of differentiated or partially differentiated cells, with the methods comprising culturing the differentiated or partially differentiated cells on a cell culture surface that comprises the cell support compositions of the present invention. In one embodiment, the cell support composition is polymerized prior to culturing the differentiated or partially differentiated cells on the cell surface In one embodiment, the differentiated or partially differentiated cells that are attached to cell culture surfaces coated with the cell support compositions of the present invention and are able to maintain their state of differentiation after attachment. Specifically, differentiated or partially differentiated cells attached on surfaces coated the cell support compositions of the present invention can maintain their state of differentiation for up to at least 2, 3, 4, 5, 6, 7, 8, 9, 10, 11, 12, 13, 14, 15, 20, 25, 30, 35, 40, 45, 50, 55, 60, 65, 70, 75, 80, 85, 90, 95 or 100 passages or even more. As used herein, “state of differentiation” is used to mean the development state of the cells after division. Maintaining a state of differentiation means that the daughter cells are at least as differentiated, and perhaps more so, than the parent cell. The differentiated or partially differentiated cells may or may not divide when cultured on surfaces coated with the cell support compositions of the present invention.

In one embodiment, neuronal precursors are seeded onto cell culture surfaces comprising the cell support compositions of the present invention. After adhesion, the cells will extend neurites and express neuron-specific genes in the process of neuronal differentiation. In another embodiment, endothelial cells are seeded are seeded onto cell culture surfaces comprising the cell support compositions of the present invention. After adhesion, the cells will associate to form branching tubular structures with lumens resembling blood vessels. These processes may or may not be inhibited in the presence of excess free peptides derived from the laminin binding sites like IKVAV.

In one embodiment, neuronal precursors are seeded onto cell culture surfaces comprising the cell support compositions of the present invention. After adhesion, the cells will extend neurites and express neuron-specific genes in the process of neuronal differentiation. In another embodiment, endothelial cells are seeded are seeded onto cell culture surfaces comprising the cell support compositions of the present invention. After adhesion, the cells will associate to form branching tubular structures with lumens resembling blood vessels. These processes may or may not be inhibited in the presence of excess free peptides derived from the laminin binding sites like IKVAV.

The cells may be from an established cell line, or they may be primary cells or genetically engineered cells. For example, neovascularization can be stimulated by angiogenic and growth-promoting factors, administered as peptides, proteins or as gene therapy. Angiogenic agents can be incorporated into the cell support compositions by culturing on the cell support compositions (or adding genetically engineered cells to unpolymerized cell support compositions) genetically engineered cells that are engineered to produce, for example, VEGF. Alternatively, where neovascularization is not desired, antiangiogenic agents can be incorporated into the cell support compositions by culturing on the cell support compositions (or adding genetically engineered cells to unpolymerized cell support compositions) genetically engineered cells that are engineered to produce, for example, angiostatin.

The term “culture surface” as used herein means a surface on which cells or tissue can be cultured. The culture surface may, for example, be glass or plastic, or polymers such as alginate. Other surfaces include but are not limited to, biopolymers such as PLAGA (polylactic acid glycolic acid copolymer), PLA (polylactic acid), PCL (polycaprolactone), PDO (polydione), PEO (polyester), metals such as but not limited to, stainless steel or titanium, glass, other forms of plastic besides polystyrene, such as but not limited to polycarbonate, polyallomer, polyethylene, and any composition of calcium phosphate crystal including but not limited to hyaluronic acid. Culture surfaces include, but are not limited to, single and multiwall culture plates, chambered and multi-chambered culture slides, cover-slips, cups, flasks, tubes, bottles, roller bottles, spinner bottles, perfusion chambers, bioreactors, fermenters and the like.

In one embodiment, the cell support compositions of the present invention can be fabricated into a scaffold, for cell culture or tissue engineering applications. For example, the cell support compositions of the present invention can be fabricated into nanofibers and mesh through electrospinning technologies as described in US2010/0120115, which is incorporated by reference. The term nanofiber as used herein means a fiber comprising a diameter of about 1000 nanometers or less. Once fabricated into a scaffold, the cell support compositions of the present invention can be placed into cell culture environment as disclosed herein.

The cell support compositions of the present invention can be used in in vivo methods as well. For example, in one embodiment of the present invention, the cell support composition can be used as a tissue regenerative composition for repair or replacement of tissues in an in vivo setting. Thus the present invention provides therapeutic methods, with the methods comprising administering the cell support compositions of the present invention to a subject in need of such therapy. The cell support compositions can be used in a variety of tissues or organs, including but not limited to, cardiac tissue, bone tissue, ligament tissue, tendon tissue, skin, muscle tissue, vasculature, liver tissue, lung tissue, and the like. The tissue to which the cell support compositions can be applied may be diseased, normal, damaged or even dead. The tissues or organs to which the cell support compositions can be applied would include all compartments or subdivisions of the tissue or organ. For example, cardiac tissue includes, but is not limited to, diseased, damaged, or missing heart tissue including myocardium, epicardium, endocardium, pericardium. For example, the cell support compositions of the present invention can be fluidized or maintained as a fluid, powderized, or pulverized and applied to or injected into or adjacent to diseased or defective cardiac tissue to promote tissue repair.

As used herein, the term “administer” and “administering” are used to mean introducing at least one compound or composition into a subject. When administration is for the purpose of treatment, the substance is provided at, or after the diagnosis of an abnormal condition, such as an infarction.

The cell support compositions may also be coadministered with other compounds or compositions. As used herein, the term “coadminister” is used to mean that each of at least two compounds are administered during a time frame wherein the respective periods of biological activity overlap. Thus the term includes sequential as well as coextensive administration of the compositions of the present invention. If more than one substance is coadministered, the routes of administration of the two or more substances need not be the same. The scope of the invention is not limited by the identity of the substance which may be coadministered with the compositions of the present invention.

For embodiments in which the cell support compositions are used in vivo, the cell support compositions can further comprise one or more therapeutic molecules including, without limitation, any pharmaceutical or drug. Examples of pharmaceuticals include, but are not limited to, anesthetics, hypnotics, sedatives and sleep inducers, antipsychotics, antidepressants, antiallergics, antianginals, antiarthritics, antiasthmatics, antidiabetics, antidiarrheal drugs, anticonvulsants, antigout drugs, antihistamines, antipruritics, emetics, antiemetics, antispasmodics, appetite suppressants, neuroactive substances, neurotransmitter agonists, antagonists, receptor blockers and reuptake modulators, beta-adrenergic blockers, calcium channel blockers, disulfuram and disulfuram-like drugs, muscle relaxants, analgesics, antipyretics, stimulants, anticholinesterase agents, parasympathomimetic agents, hormones, anticoagulants, antithrombotics, thrombolytics, immunoglobulins, immunosuppressants, hormone agonists/antagonists, vitamins, antimicrobial agents, antineoplastics, antacids, digestants, laxatives, cathartics, antiseptics, diuretics, disinfectants, fungicides, ectoparasiticides, antiparasitics, heavy metals, heavy metal antagonists, chelating agents, gases and vapors, alkaloids, salts, ions, autacoids, digitalis, cardiac glycosides, antiarrhythmics, antihypertensives, vasodilators, vasoconstrictors, antimuscarinics, ganglionic stimulating agents, ganglionic blocking agents, neuromuscular blocking agents, adrenergic nerve inhibitors, anti-oxidants, vitamins, cosmetics, anti-inflammatories, wound care products, antithrombogenic agents, antitumoral agents, antiangiogenic agents, anesthetics, antigenic agents, wound healing agents, plant extracts, growth factors, emollients, humectants, rejection/anti-rejection drugs, spermicides, conditioners, antibacterial agents, antifungal agents, antiviral agents, antibiotics, tranquilizers, cholesterol-reducing drugs, antitussives, histamine-blocking drugs, monoamine oxidase inhibitor. All substances listed by the U.S. Pharmacopeia are also included within the substances of the present invention.

The cell support compositions of the present invention may or may not further comprise objects. Examples of objects include, but are not limited to, tablets, vesicles, liposomes, capsules, nanoparticles, and other structures that could enclose molecules. In some embodiments, the additional objects comprise vesicles, liposomes, capsules, or other enclosures that contain compounds that are released at a time such as at the time of implantation or upon later stimulation or interaction. In one illustrative embodiment, transfection agents such as liposomes contain desired nucleotide sequences that can be incorporated into cells that are located in or on the cell support compositions.

The cell support compositions of the present invention may or may not further comprise additional amino acids, proteins or peptides that are not necessarily signaling molecules. Examples include, but are not limited to, structural proteins, enzymes, and peptide hormones. These additional proteins and peptides compounds can serve a variety of functions. In some embodiments, the matrix may contain peptides containing a sequence that suppresses enzyme activity through competition for the active site. In other applications, antigenic agents that promote an immune response and invoke immunity can be incorporated into a construct.

The cell support compositions of the present invention may or may not further comprise additional nucleic acids. Examples of nucleic acids include, but are not limited to deoxyribonucleic acid (DNA), ent-DNA, oligonucleotides, aptamers, and ribonucleic acid (RNA). Embodiments involving DNA include, but are not limited to, cDNA sequences, natural DNA sequences from any source, and sense or anti-sense oligonucleotides. For example, DNA can be naked (e.g., U.S. Pat. Nos. 5,580,859; 5,910,488) or complexed or encapsulated (e.g., U.S. Pat. Nos. 5,908,777; 5,787,567). DNA can be present in vectors of any kind, for example in a viral or plasmid vector. In some embodiments, nucleic acids used will serve to promote or to inhibit the expression of genes in cells inside and/or outside the present composition. The nucleic acids can be in any form that is effective to enhance uptake into cells.

The present invention also provides methods of preparing basement membrane extracts that has been isolated from cardiac or smooth muscle tissue from an organism. As used herein, isolated means that the material has been removed from its native environment, regardless of the level of impurities that may or may not be present. For example, cardiac basement membrane has been isolated when it has been removed or extracted from cardiac tissue. As used herein, the isolated material can be placed back into a setting that resembles, substantially resembles or is identical to the original environment from which the material was isolated. For example, the isolated cardiac basement membrane extract may be inserted or introduced back into cardiac tissue of another organism. Any degree of purification or concentration greater than that which occurs in the natural setting of the compound, including, but not limited to, (1) purification from other associated structures or compounds or (2) association with structures or compounds to which the material is not normally associated is included in the term purified.

The following examples are illustrative and are not intended to limit the scope of the invention described herein.

### EXAMPLES

### Example 1

### Process for Isolating Basement Membrane Components from Cardiac Muscle

Methods for preparing the isolated human basement membrane extract (HBME) can be conducted at a temperature of about 0° C., 4° C., 15° C., 24° C., 27° C., 30° C., or 37° C. The solutions used for the isolation process can be chilled or warmed to the appropriate temperature before starting the process.

Frozen, thinly sliced or minced pieces of cardiac tissue were washed or not washed with a buffer such as phosphate or potassium or calcium chloride at a concentration such as 0.4M to 0.05M for a period of time such a 1,2,3,4, . . . 24 hour. After washing ice cold 3.0 M, 3.1, 3.2, 3.3, 3.4, 3.5, 3.6, 3.7, 3.8, 3.9, 4.0 NaCl or KCl buffer is added to tissue including protease inhibitors such as PMSF, Aprotinin, leupeptin, pepstatin A, NEM, phenanthroline, Benzamidine, AEBSF, Bestatin, E-64 (volume to weight 2:1, 3:1, 4:1, 5:1, 6:1, 7:1) and homogenized.

The homogenized slurry was then centrifuged and the supernatant was poured off and the pellets collected in a tripour beaker. Another aliquot of 3.0 M, 3.1, 3.2, 3.3, 3.4, 3.5, 3.6, 3.7, 3.8, 3.9, 4.0 NaCl or KCl buffer was added as above and the pellets were washed and homogenized. After homogenization, the slurry was again centrifuged. These washes and homogenizations were repeated 3-5 times.

After repeated washing of the processed tissue, 2 M Urea Buffer was added at a concentration of 1 ml/gm starting weight of tissue. The mixture was stirred overnight. The mixture was then centrifuged and decanted. The supernatant was saved for later use. The pellet was recovered and homogenized again in 2 M urea buffer (half the volume as the first use of the Urea Buffer) stirred at least 2 hours.

The mixture was centrifuged again and the supernatant was recovered and added to the first volume of supernatant recovered above. The mixture was dialysed against a buffer such as phosphate, TEA, Bicine, MOPS, Tris, Hepes, TAPSO, PIPES, MES, or other Good's buffers. The last buffer used can be DMEM, DMEM/F12, HEPES, Hanks or other cell culture media. Antibiotics can be added to the extract and stored at 4° C. or long term at −80° C. The concentration of the mixture is determined by dialyzing the mixture against dH₂O and lyophilizing in tared containers and weighing when dry.

### Example 2

### Coating Cell Culture Surfaces with Isolated HMBE

The protein needed to coat the plate at each concentration is calculated at about 10-20 μg/cm². Other supplemental purified matrix molecules are coated in the range of 5-20 μg/cm². MATRIGEL™ is coated at 17.5 μg /cm² according to the WiCell protocol and the cell support composition product may be used at this concentration or less for some applications or at higher concentration for other applications.

For dry coating, 100 μl of the cell support composition was pipetted into each well of plate at the desired concentration and allowed to dry uncovered in biosafety cabinet. After coating, the plates were washed very gently with 1-2 volumes of a typical cell culture buffer such as culture media, PBS or Hanks balanced salt solution.

After washing, 200 μl of sterile buffer containing 2% albumin can be added to each well and incubated for 15 min room temperature. After incubation, the albumin mixture is removed and media is added to wash the plate.

### Example 3

### Cell Attachment Assay

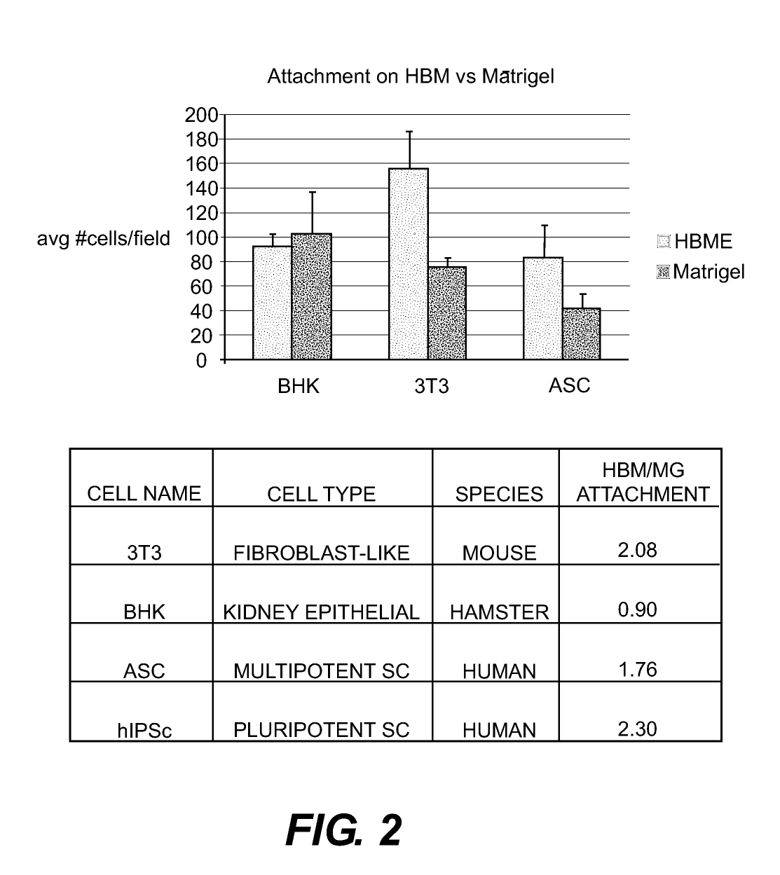

Plates were coated at various concentrations of the cell support compositions of the present invention (see FIG. 2) to test cell attachment using 3T3, BHK and adipose stem cells. Cells were allowed to attach to plates coated with the indicated amounts of the cell support compositions of the present invention for 60 minutes in the absence of serum. After attachment, the plates were gently rinsed to remove unattached cells and attached cells were counted. Similar results were observed with human mesenchymal stem cells.

### Example 4

### Differentiation Assays of Attached Cells

Adipose derived stem cells are isolated using well known procedures and are plated at a density of about 5,000 cells/100 mm dish. The dishes have been coated with isolated the cell support compositions of the present invention, and the cells are cultured for a few days after plating. After successive rounds of cell division, some clones are picked with a cloning ring and transferred to wells in a 48 well plate, where the wells have also been coated with isolated the cell support compositions of the present invention. These cells are cultured for several weeks, changing the medium twice weekly, until they are about 80% to about 90% confluent (at 37° C., in about 5% CO₂ in ⅔ F₁₂ medium (with 20% fetal bovine serum) and ⅓ standard medium). Thereafter, each culture is transferred to a 35 mm dish (coated with the cell support compositions of the present invention) and grown, and then retransferred to a 100 mm dish (coated with isolated the cell support compositions of the present invention) and grown until close to confluence. Following this, one cell population is frozen, and the remaining populations were plated on 12 well plates, at 1000 cells/well.

The cells are cultured for more than 15 passages in medium and on surfaces coated with the cell support compositions of the present invention and monitored for signs of differentiation. The undifferentiated state of each clone remains true after successive rounds of division.

Populations of the clones are then established on culture surfaces not coated with the cell support compositions of the present invention and then exposed to adipogenic, chondrogenic, myogenic, and osteogenic medium, or other medium or factors known to promote differentiation of the stem cells. The clones are able to differentiate into bone, fat, cartilage, and muscle when exposed to the respective media as determined by phonotypic assays of protein expression and visual inspection of cell types.

### Example 5

### SDS-PAGE Analysis of HMBE

An SDS-PAGE was performed on the cell support compositions of the present invention using standard procedures. The gel showed that the cell support compositions of the present invention is an aggregate of macromolecules purified from human cardiac tissue. These polypeptides range in size between 350 and 100 killodaltons (kDa) and cross-react with antibodies against mouse and human laminin, type IV collagen and heparan sulfate proteoglycan.

### Example 6

### Process for Isolating Basement Membrane Components from Smooth Muscle

Frozen, thinly sliced or minced pieces of smooth muscle tissue are washed with buffers such as Phosphate or potassium or Calcium Chloride then added to cold 3.4M NaCl or KCl buffer (3× volume to weight) including protease inhibitors such as PMSF, Aprotinin, leupeptin, pepstatin A, NEM, phenanthroline, Benzamidine, AEBSF, Bestatin, E-64 and homogenized.

The homogenized slurry is then centrifuged and the supernatant is poured off and the pellets collected. Another aliquot of 3.4M NaCl buffer is added as above and the pellets are homogenized. After homogenization, the slurry is again centrifuged. These homogenizations are repeated 4-5 times.

After repeated washing by homogenization of the processed tissue, 2 M Urea Buffer is added at a concentration of 1 ml/gm starting weight of tissue. The mixture is stirred overnight. The mixture is then centrifuged and decanted. The supernatant is saved for later use. The pellet is recovered and homogenized again in 2 M urea buffer (half the volume as the first use of the Urea Buffer) stirred at least 2 hours.

The mixture is centrifuged again and the supernatant is recovered and added to the first volume of supernatant recovered above. Antibiotics can be added to the combined supernatant and stored at 4° C. or long term at −80° C.

### Example 7

### Direct Injection of the Cell Support Composition into a Subject

The cell support composition as prepared according to Example 1 is prepared for injection into damaged cardiac tissue. A procedure is administered to mice to induce myocardial infarction as disclosed in Salto-Tellez, M. et al., Cardiovascular Path., 13(2):91-97 (2004). Specifically, a coronary artery is ligated to cause infarction. After reperfusion of the vessel, a cell support composition of the present invention is injected into the damaged cardiac tissue.

The injections can be single injections or multiple injections over time. A single injection of the cell support composition of the present invention improves heart function as measured by normal parameters, such as ejection volume, end systolic volume, stroke volume and echocardiagraphy. In as little as 7 days, the mice receiving the cell support composition of the present invention show improvement over control animals not receiving the compositions of the present invention.

What is claimed is:

1. A cell support composition comprising in parts by weight 5-50% laminin, wherein laminin type 1 and laminin type 3 are not present in any amount, 5-50% collagen, 1-10% nidogen, 5-60% heparan sulfate proteoglycan, and 1-10% entactin.

2. The cell support composition of claim 1, wherein laminin type 5 is not present in any amount.

3. The cell support composition of claim 1, wherein collagen type IV is present.

4. The cell support composition of claim 1, wherein collagen type I is present.

5. The cell support composition of claim 1, wherein collagen type VI is present.

6. The cell support composition of claim 3, wherein collagen type VI is present.

7. The cell support composition of claim 4, wherein collagen type VI is present.

8. The cell support composition of claim 7, wherein collagen type VI is present.

9. The cell support composition of claim 1, wherein the heparan sulfate proteoglycan comprises perlecan, wherein the perlecan is present in the cell support composition in an amount from about 5% to about 60% by weight.

10. The cell support composition of claim 1, further comprising titan protein.

11. The cell support composition of claim 1, further comprising myosin 7.

12. The cell support composition of claim 1, further comprising actinin protein.

13. The cell support composition of claim 1, wherein the cell support composition promotes the maintenance of plasticity of stem cells in cell culture.

14. The cell support composition of claim 1, wherein the cell support composition polymerizes at a temperature of at least about 37° C. within about 1 hour.

15. The cell support composition of claim 1, wherein the cell support composition is polymerized.

16. The cell support composition of claim 15, wherein the polymerized cell support composition is in form of nanofibers.

17. A cell culture surface comprising the cell support composition of claim 15.

18. A method of maintaining the plasticity of stem cells, comprising culturing the stem cells on a cell culture surface comprising the cell support composition of claim 1, wherein the cell support composition has been polymerized prior to culturing the stem cells thereon.

19. The method of claim 18, wherein the stem cells are adult stem cells (ASCs).

20. The method of claim 18, wherein the stem cells are embryonic stem cells (ESCs).

21. The method of claim 18, wherein the stem cells are induced pluripotent cells (iPCs).

22. The method of claim 18, wherein the cell support composition is in the form of nanofibers.

23. A method of treating tissue comprising administering to a subject in need of treatment the cell support composition of claim 1.
